(19) United States
(12) Patent Application Publication
(10) Pub. No.: US 2009/0227673 A1
(43) Pub. Date: Sep. 10, 2009

(54) Method and Composition for the Treatment of Parkinson's Disease

(76) Inventor: Bao Ting Zhu, Lexington, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 12/087,183

(22) PCT Filed: Jan. 12, 2007
(86) PCT No.: PCT/US2007/000854

§ 371 Date: Jan. 15, 2009

Related U.S. Application Data
(60) Provisional application No. 60/759,111, filed on Jan. 13, 2006.

(51) Int. Cl. A61K 31/216; A61K 31/195; A61P 25/16

(52) U.S. Cl. 514/543; 514/567

(57) ABSTRACT

It has been discovered that polyphenols are effective as a co-pharmaceutical in combination with traditional dual drug therapies of catecholamines and decarboxylase inhibitors for the treatment of Parkinson's disease. Accordingly, a method of treating Parkinson's disease comprising administering to a subject suffering from Parkinson's disease a pharmaceutical composition comprising at least one catecholamine, at least one decarboxylase inhibitor, and at least one polyphenol is provided.

### BACKGROUND OF THE INVENTION

The present invention relates generally to the treatment of Parkinson's disease. More specifically, the invention relates to the use of dietary compounds in the treatment of Parkinson's disease.

Parkinson's disease is a degenerative disorder of the central nervous system that often impairs the sufferer's motor skills and speech. Parkinson's disease belongs to a group on conditions commonly referred to as movement disorders. The disease is characterized by muscle rigidity, tremor, a slowing of physical movement, and in extreme cases, a loss of physical movement. The primary symptoms are the results of excessive muscle contraction, normally caused by the insufficient formation and action of dopamine, which is produced in the dopaminergic neurons of the brain. Secondary symptoms may include high level cognitive dysfunction and subtle language problems. Parkinson's disease is both chronic and progressive.

There are currently no blood or laboratory tests that have been proven to help in diagnosing Parkinson's disease. The diagnosis, therefore, is based on medical history and a neurological exam. The disease can be difficult to diagnose accurately. The United Parkinson's Disease Rating Scale is the primary clinical tool used to assist in diagnosis and to determine severity of Parkinson's. Indeed, only 75% of clinical diagnoses of Parkinson's are confirmed by autopsy. Early signs and symptoms of Parkinson's may sometimes be dismissed as the effects of normal aging. Physicians may need to observe a person for some time before it is apparent that the symptoms are consistently present. Usually, doctors look for shuffling of feet and lack of swing in the arms. Doctors may sometimes request brain scans or laboratory tests in order to rule out other diseases. CT and MRI brain scans of people with Parkinson's usually appear normal.

Parkinson's disease is widespread, with a prevalence estimated between about 100 and 250 cases per 100,000 in North America, and 1.7 per hundred in China. Because prevalence rates can be affected by socio-economically driven differences in survival as well as biased by survey technique problems, incidence is a more sensitive indicator with rates to a high of 14.8 per 100,000 in Finland. Incidence has been estimated by several groups. One study observed an age and sex corrected incidence of 13.4 per 100,000/year. The study noted a rapid increase in incidence with age, male rates nearly double female rates, and an elevated rate among Hispanics. Another study (of a population of people aged 65 to 85) calculated incidence, adjusted for age and ex, of 186.8 per 100,000 per year, with men's rates being 2.55 times that of women.

Cases of Parkinson's are reported at all ages, though it is quite rare in people younger than 40 and the average age at which symptoms begin is 58-60. It is principally a disease of the elderly. It occurs in all parts of the world, but appears to be more common in people of European ancestry than in those of African ancestry. Those of East Asian ancestry have an intermediate risk. It is more common in rural than urban areas.

The symptoms of Parkinson's disease result from the loss of pigmented dopamine-releasing (dopaminergic) cells and subsequent loss of melanin, secreted by the same cells, in the pars compacta region of the substantia nigra (literally “black substance”). These neurons project to the striatum and their loss leads to alterations in the activity of the neural circuits within the basal ganglia that regulate movement, in essence an inhibition of the direct pathway and excitation of the indirect pathway.

The direct pathway facilitates movement and the indirect pathway inhibits movement, thus the loss of these cells leads to a hypokinetic movement disorder. The lack of dopamine results in increased inhibition of the ventral lateral nucleus of the thalamus, which sends excitatory projections to the motor cortex, thus leading to hypokinesia.

There are four major dopamine pathways in the brain; the nigrostriatal pathway, referred to above, mediates movement and is the most conspicuously affected in early Parkinson's disease. The other pathways are the mesocortical, the mesolimbic, and the tuberoinfundibular. These pathways are associated with, respectively: volition and emotional responsiveness; desire, initiative, and reward; and sensory processes and maternal behavior. Disruption of dopamine along the non-striatal pathways likely explains much of the neuropsychiatric pathology associated with Parkinson's disease.

The mechanism by which the brain cells in Parkinson's are lost may consist of an abnormal accumulation of the protein alpha-synuclein bound to ubiquitin in the damaged cells. The alpha-synuclein-ubiquitin complex cannot be directed to the proteosome. This protein accumulation forms proteinaceous cytoplasmic inclusions called Lewy bodies. Recent research on pathogenesis of disease has shown that the death of dopaminergic neurons by alpha-synuclein is due to a defect in the machinery that transports proteins between two major cellular organelles—the endoplasmic reticulum (ER) and the Golgi apparatus. Certain proteins like Rabi may reverse this defect caused by alpha-synuclein in animal models http://en.wikipedia.org/wiki/Parkinson%27s disease—note-16# note-16

Excessive accumulations of iron, which are toxic to nerve cells, are also typically observed in conjunction with the protein inclusions. Iron and other transition metals such as copper bind to neuromelanin in the affected neurons of the substantia nigra. So, neuromelanin may be acting as a protective agent. Alternately, neuromelanin (an electronically active semiconductive polymer) may play some other role in neurons. That is, coincidental excessive accumulation of transition metals, etc. on neuromelanin may figure in the differential dropout of pigmented neurons in Parkinsonism. The most likely mechanism is generation of reactive oxygen species.

Iron induces aggregation of synuclein by oxidative mechanisms Similarly, dopamine and the byproducts of dopamine production enhance alpha-synuclein aggregation. The precise mechanism whereby such aggregates of alpha-synuclein damage the cells is not known. The aggregates may be merely a normal reaction by the cells as part of their effort to correct a different, as-yet unknown, insult. Based on this mechanistic hypothesis, a transgenic mouse model of Parkinson's has been generated by introduction of human wild-type α-synuclein into the mouse genome under control of the platelet-derived-growth factor-β promoter.

The most widely-used form of treatment for Parkinson's is oral administration of levodopa (L-DOPA). L-DOPA is transformed into dopamine in the dopaminergic neurons by L-aromatic amino acid decarboxylase (also commonly referred to as “dopa-decarboxylase”). Only 1-5% of L-DOPA enters the dopaminergic neurons due to rapid peripheral metabolism. Specifically, the drug is rapidly decarboxylated by dopa-decarboxylase and also o-methylated by catechol-methyltransferase (COMT) inhibitor in peripheral tissues (FIG. 1). Accordingly, less than 1% of the L-DOPA reaches the central nervous system for biosynthesis of dopamine and to exert its therapeutic effects.

In practice, L-DOPA is typically co-administered with a decarboxylase inhibitor, such as carbidopa or benserazide. More recently, a COMT inhibitor, such as entacapone or tolcapone, has been added to the treatment.

Carbidopa and benserazide are dopa decarboxylase inhibitors. They help to prevent the metabolism of L-DOPA before it reaches the dopaminergic neurons and are generally given as combination preparations of carbidopa/levodopa (co-careldopa) (e.g. Sinemet, Parcopa) and benserazide/levodopa (co-beneldopa) (e.g. Madopar). There are also controlled release versions of Sinemet and Madopar that spread out the effect of the L-DOPA. Duodopa is a combination of levodopa and carbidopa, dispersed as a viscous gel. Using a patient-operated portable pump, the drug is continuously delivered via a tube directly into the upper small intestine, where it is rapidly absorbed.

More recent studies showed that when the decarboxylase is chronically inhibited, the majority of the surplus L-DOPA is metabolized preferably by peripheral COMT, resulting in the formation of 3-methoxy-DOPA and, therefore, maintaining a reduced level of L-DOPA reaching the central nervous system. Addition of a COMT inhibitor, to form a three-pharmaceutical composition, is strongly recommended for clinical use to replace the traditional double pharmaceutical composition (L-DOPA+decarboxylase inhibitor).

Talcapone and entacapone are COMT inhibitors that have been shown to inhibit the COMT enzyme, thereby prolonging the effects of L-DOPA, and have been used to complement L-DOPA. Such COMT inhibitors could reduce the formation of 3-methoxy-DOPA from L-DOPA. Consequently, the bioavailability of L-DOPA would be improved, its entry to the brain increased, and its half-life prolonged. These effects have been observed consistently in animal models as well as in normal human volunteers or Parkinson's patients treated with entacapone and tolcapone.

These compounds, however, contain a potentially toxic/carcinogenic nitrocatechol structure. In late 1998, the marketing of tolcapone was suspended in the European Union and Canada due to serious adverse reactions. In the United States, because of serious concerns over its potential toxicity, tolcapone is only used as an adjunct in Parkinson's patients who already receive the L-DOPA and carbidopa combination but still experience symptom fluctuations.

It would be desirable, therefore, to develop replacements for the COMT inhibitors that exhibit the same beneficial properties, while avoiding the dangerous side-effects of the known COMT inhibitors.

### SUMMARY OF THE INVENTION

Briefly, therefore, the present invention is directed to a method for treating Parkinson's disease. The method includes administering to a subject L-DOPA, at least one decarboxylase inhibitor, and at least one polyphenol.

In another aspect, the present invention is directed to a pharmaceutical composition for sufferers of Parkinson's disease. The pharmaceutical composition includes L-DOPA, at least one decarboxylase inhibitor, and at least one polyphenol.

### BRIEF DESCRIPTION OF THE DRAWINGS

FIG. 1 is a representative reaction scheme of COMT-mediated o-methylation of L-DOPA, norepinephrine, and epinephrine, as well as chemical structures of compounds referenced herein.

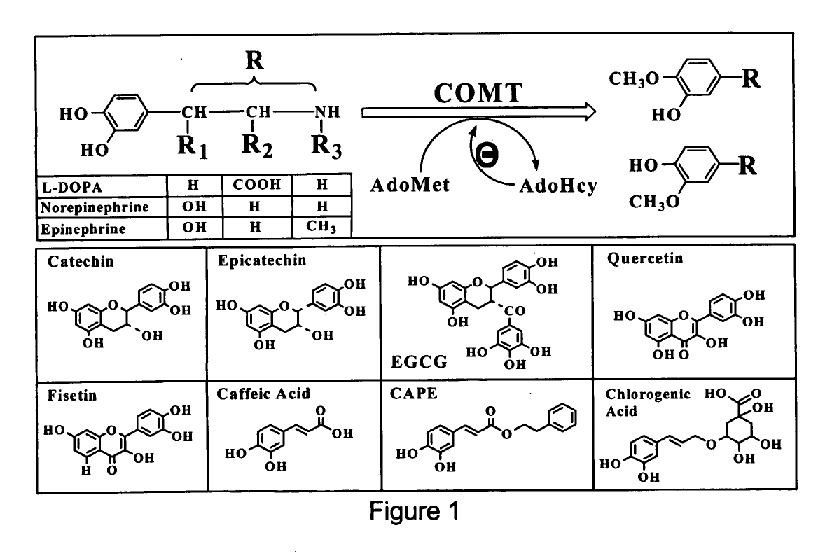

FIG. 2 demonstrates the metabolism rates of catecholamines in the presence of catechin, epicatechin, and EGCG.

FIG. 3 demonstrates the metabolism rates of catecholamines in the presence of tea extracts.

FIG. 4 demonstrates the metabolism rates of catecholamines in the presence of quercetin and fisetin.

FIG. 5 demonstrates the metabolism rates of catecholamines in the presence of coffee extracts.

### DETAILED DESCRIPTION OF SEVERAL EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In accordance with the present invention, it has been discovered that polyphenols are effective as a co-pharmaceutical in combination with traditional dual drug therapies of catecholamines and decarboxylase inhibitors for the treatment of Parkinson's disease. Accordingly, in one aspect, the present invention is a method of treating Parkinson's disease comprising administering to a subject suffering from Parkinson's disease a pharmaceutical composition comprising at least one catecholamine, at least one decarboxylase inhibitor, and at least one polyphenol.

In another aspect, the present invention is a pharmaceutical composition for treating Parkinson's disease. The pharmaceutical composition includes at least one catecholamine, at least one decarboxylase inhibitor, and at least one polyphenol.

In accordance with the present Invention, it has been discovered that polyphenols may serve as effective COMT inhibitors. More specifically, the polyphenols used in accordance with the present method and combination are strong inhibitors of human COMT-mediated o-methylation metabolism of catecholamines, including L-DOPA. Beneficially, these polyphenols, often already present to some degree in the daily diet of most humans, have little or no toxicity. Accordingly, they demonstrate the positives of the previously used COMT inhibitors without the potentially, and well-documented, side effects when used to treat Parkinson's disease.

In the present method, a subject in need of prevention or treatment of Parkinson's disease is treated with an amount of at least one catecholamine, an amount of at least one decarboxylase inhibitor and an amount of at least one polyphenol, where the amount of the catecholamine, the at least one decarboxylase inhibitor, and the at least one polyphenol, when administered together, either as discreet components, as an admixed composition, and combinations thereof, provide a dosage or amount of the combination that is sufficient to constitute a Parkinson's disease treatment amount sufficient to treat the symptoms and underlying disease.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding or reducing adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

In the present method, the amount of catecholoamine and decarboxylase inhibitors that are used should be similar to those recognized as a therapeutically acceptable amount by those having ordinary skill in the art.

Suitable catecholamines contemplated as useful in conjunction with the present invention include dopamine, epinephrine, and norepinephrine. Of these, L-DOPA is particularly preferred. For ease of discussion, the invention will be described with reference to L-DOPA. Those having ordinary skill in the art will recognize that, as used herein, L-DOPA may include other catecholamines, unless the application explicitly, or by context, implies otherwise.

Suitable decarboxylase inhibitors contemplated as useful in conjunction with the present invention are those recognized as useful in the art in the treatment of Parkinson's disease. Especially preferred decarboxylase inhibitors for use in accordance with the present invention include one or both of carbidopa and benserazide.

The amount of polyphenol that is used in the subject method may be an amount that, when administered with the L-DOPA and decarboxylase inhibitor, is sufficient to reduce the rate of o-methylation of the L-DOPA to a rate that will improve the bioavailability of L-DOPA for treatment of Parkinson's. In the present method, the amount of polyphenol that is used in the novel method of treatment preferably ranges from about 1 to about 100 milligrams per day per kilogram of body weight of the subject (mg/day·kg), more preferably from about 5 to about 50 mg/day·kg.

Those having ordinary skill in the art will recognize that polyphenols are a group of chemicals typically found in plants and characterized by the presence of more than one phenol group per molecule. Polyphenols are further categorized into tannins, lignins, and favonoids. Notable sources of polyphenols include berries, tea, wine, olive oil, chocolate/cocoa, pomegranates, walnuts, peanuts, yerba mate, grapes, and other fruits and vegetables.

Dietary polyphenols are considered especially suitable for use in accordance with the present invention. More preferred are catechins. Those having ordinary skill in the art will recognize that catechins are polyphenolic antioxidant plant metabolites. Catechins are often referred to as flavonoids or bioflavonoids (these terms are interchangeable in the art because flavonoids are biological in origin, and will be used herein in the same manner).

Notable natural sources of flavonoids include all citrus fruits, grapes, berries, onions, parsley, legumes, green tea, black tea, red wind, seabuckthorn, and dark chocolate. Citrus bioflaovonoids include hesperidin, quericeten, rutin, and tangeritin. The primary tea flavonoids are the catechins (catechin, epicatechin, epicatechin gallate, and epigallocatechin gallate). Grape skins contain significant amounts of flavonoids, as well as other polyphenols. Both red and white wine contain flavonoids, however, since red wine is produced by fermentation in the presence of the grape skins, red wine has been observed to contain higher levels of flavonoids.

Flavonoids are most commonly synthesized by the phenylpropanoid pathway, in which the amino acid phenylalanine is used to produce 4-coumaryl-CoA. This can be combined with malonyl-DcO to yield the true backbone of flavonoids, a group of compounds called chalcones. Ring-closure of these compounds results in the familiar form of flavonoids, a three-ringed structure. The metabolic pathway continues through a series of enzymatic modifications to yield flavanones, dihydroflavonols, and, then, anthocyanins. Along this pathway many products can be formed including the flavonols, flavan-3-ols, proanthyocyanidins (tannins) and a host of other polyphenolics.

As stated above, catechins are especially preferred in the method of the present invention. Catechins include catechin, epicatechin, epigallocatechin, and catechin gallates. Catechin and epicatechin are epimers, with (−) epicatechin and (+) catechin being the most common optical isomers found in nature. Epigallocatechin contains an additional phenolic hydroxyl group when compared to epicatechin, similar to the difference between pyrocatechol and pyrogallol. Catechin gallates are gallic acid esters of the catechins; such as EGCG (epigallocatechin gallate).

When the polyphenol comprises catechin, it is preferred that the amount used is within a range of from about 1 to about 100 mg/day·kg, and even more preferably from about 5 to about 50 mg/day·kg.

When the polyphenol comprises epicatechin, it is preferred that the amount used is within a range of from about 1 to about 100 mg/day·kg, and even more preferably from about 5 to about 50 mg/day·kg.

When the polyphenol comprises epigallocatechin, it is preferred that the amount used is within a range of from about 1 to about 100 mg/day·kg, even more preferably from about 5 to about 50 mg/day·kg.

In the present method, and in the subject compositions, the L-DOPA and decarboxylase inhibitors are administered with, or are combined with, a polyphenol. The components may be administered concurrently, sequentially, or some combination thereof.

The combination of catecholamines, decarboxylase inhibitors, and polyphenols can be supplied in the form of a novel therapeutic composition that is believed to be within the scope of the present invention. The relative amounts of each component in the therapeutic composition may be varied and may be as described just above. The components that are described above can be provided in the therapeutic composition so that the preferred amounts of each of the three components are supplied by a single dosage, a single capsule for example, or, by up to four, or more, single dosage forms.

When the novel combination is supplied along with a pharmaceutically acceptable carrier, a pharmaceutical composition is formed. A pharmaceutical composition of the present invention is directed to a composition suitable for the treatment of Parkinson's disease. The pharmaceutical composition comprises a pharmaceutically acceptable carrier and a combination including at least one catecholamine, at least one decarboxylase inhibitor, and at least one polyphenol. Pharmaceutically acceptable carriers include, but are not limited to, physiological saline, Ringers, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The method and combination of the present invention are useful for, but not limited to, the treatment Parkinson's disease and related diseases. For example, there are other disorders that are called Parkinson-plus diseases. These include multiple system atrophy, progressive supranuclear palsy, and corticobasal degeneration.

The terms “treating” or “to treat” means to alleviate symptoms, eliminate the causation either on a temporary or permanent basis, or to prevent or slow the appearance of symptoms. The term “treatment” includes alleviation, elimination of causation of, or prevention of, but not limited to, any of the diseases or disorders described above. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has Parkinson's and/or any one of the known Parkinson's plus diseases. The subject is typically a human subject.

The pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “drug therapy,” “combination therapy,” “co-administration,” “administration with,” or “co-therapy,” in defining the use of at least one catecholamine, at least one decarboxylase inhibitor, and at least one polyphenol, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

The phrase “therapeutically-effective” and “effective for the treatment, prevention, or inhibition”, are intended to qualify the amount of each agent for use in the combination therapy which will achieve the goal of improvement in symptoms and the underlying disease, and the frequency of incidence over treatment of each agent by itself, while avoiding adverse side effects typically associated with alternative therapies.

Although the combination of the present invention may include administration of each component within an effective time of each respective component, it is preferable to administer the respective components contemporaneously, and more preferable to administer the respective components in a single delivery dose.

In particular, the combinations of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n−3 polyunsaturated fatty acids may find use in the preparation of injectables;

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages. In one embodiment, it may be preferably to administer the components in a 1:1:1-10 ratio of catecholamine:decarboxylase inhibitor: polyphenol.

Various delivery systems include capsules, tablets, and gelatin capsules, for example.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples all percentages are given on a weight basis unless otherwise indicated.

### EXAMPLES

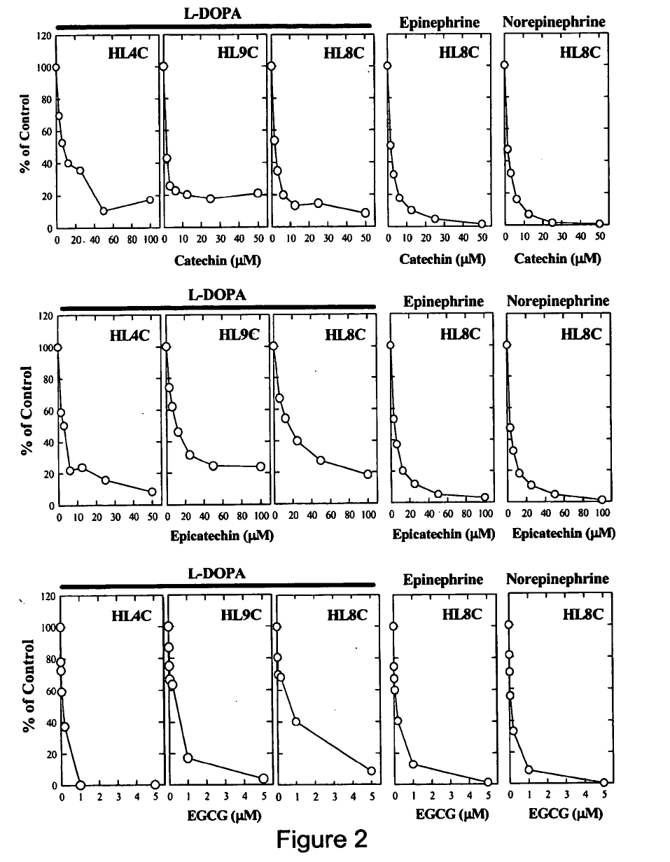

Catechins derived from tea, such as (−) epigallocatechin-3-o-gallate catechin (EGCG), catechin, and epicatechin were tested to determine their ability to inhibit human COMT-mediated o-methylation metabolism of L-DOPA and other endogenous catecholamines in a concentration-dependent manner. The half maximal inhibitory concentration (IC₅₀) values of EGCG are 0.02-0.07 μM, and the IC₅₀ values of eatechin and epicatechin are 0.5-1 μM. FIG. 2 demonstrates the inhibition of human liver COMT-mediated o-methylation of catecholamines by increasing concentrations of catechin (upper panels), epicatechin (middle panels), and EGCG (lower panels). The incubation mixture consisted of 10 μM catecholamine substrate, 250 μM [³H-methyl] S-Adenosyl-L-methionine (containing 0.2 μCI), 0.25 mg/mL of human liver cytosolic protein, 1 mM dithiothreitol, 1.2 mM MgCl₂, and a dietary inhibitor (concentration as indicated) in a final volume of 0.25 mL tris-HCL buffer (10 mM, pH 7.4). Incubations were carried out at 37° C. for 10 min. Each point is the mean of duplicate determinations. Note that a total of three different human liver cytosolic preparations (HL4C, HL9C, HL8C) were tested.

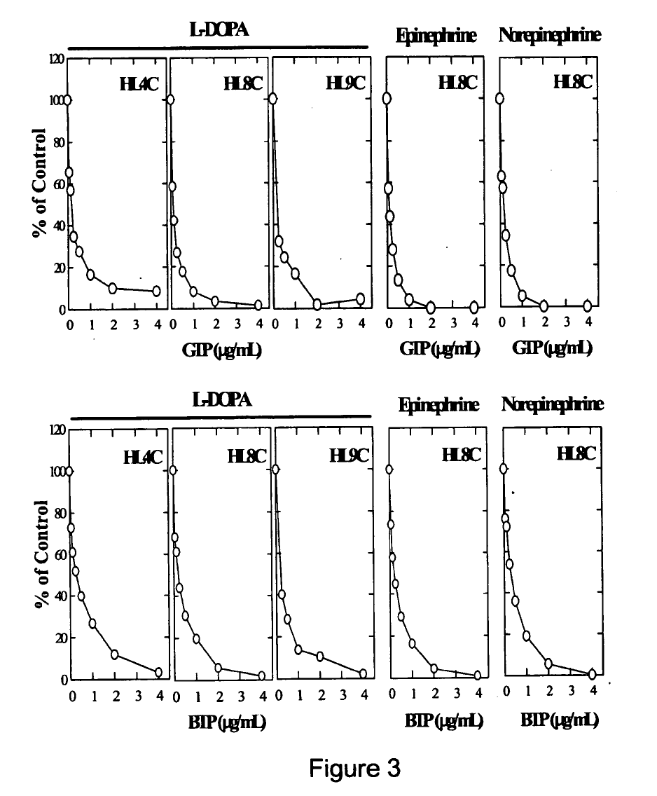

FIG. 3 demonstrates the metabolism rates for the crude extracts from green tea and black tea. As can be seen in FIG. 3, the crude extracts have high potency and efficacy for inhibiting human COMT-mediated o-methylation of L-DOPA and other catecholamines. In FIG. 3, the inhibition of human liver COMT-mediated o-methylation of catecholamines by a green tea polyphenol (GTP) extract (upper panels) and a black tea polyphenol (BTP) extract (lower panels) is demonstrated. The incubation mixture consisted of 10 μM catecholamine substrate, 250 μM [³H-methyl]AdoMet (containing 0.2 μCi), 0.25 mg/mL of human liver cytosolic protein, 1 mM dithiothreitol, 1.2 mM MgCl₂, and a dietary inhibitor (concentration as indicated) in a final volume of 0.25 mL tris-HCL buffer (10 mM, pH 7.4). Incubations were carried out at 37° C. for 10 min. Each point is the mean of duplicate determinations. Note that a total of three different human liver cytosolic preparations (HL4C, HL9C, HL8C) were tested.

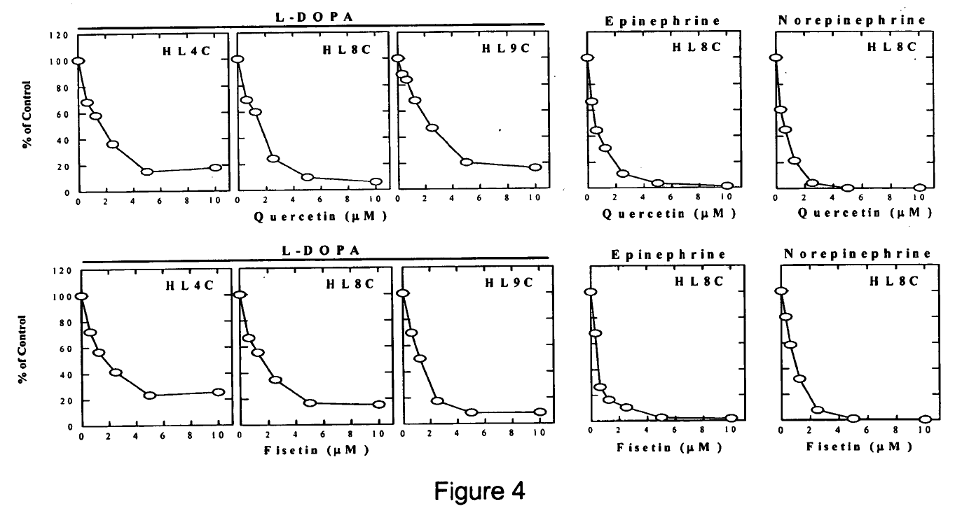

FIG. 4 represents the metabolism data of catechol-containing bioflavonoids, (such as quercetin and fisetin), which can also strongly inhibit human COMT-mediated o-methylation of L-DOPA and other catecholamines. The IC₅₀ values are ˜0.1 μg/mL. FIG. 4 demonstrates the inhibition of human liver COMT-mediated o-methylation of catecholamines by quercetin (upper panels) and fisetin (lower panels). The incubation mixture consisted of 10 μM catecholamine substrate, 250 μM [³H-methyl]AdoMet (containing 0.2 μCi), 0.25 mg/mL of human liver cytosolic protein, 1 mM dithiothreitol, 1.2 mM MgCl₂, and a dietary inhibitor (concentration as Indicated) in a final volume of 0.25 mL tris-HCL buffer (10 mM, pH 7.4). Incubations were carried out at 37° C. for 10 min. Each point is the mean of duplicate determinations. Note that a total of three different human liver cytosolic preparations (HL4C, HL9C, HL8C) were tested.

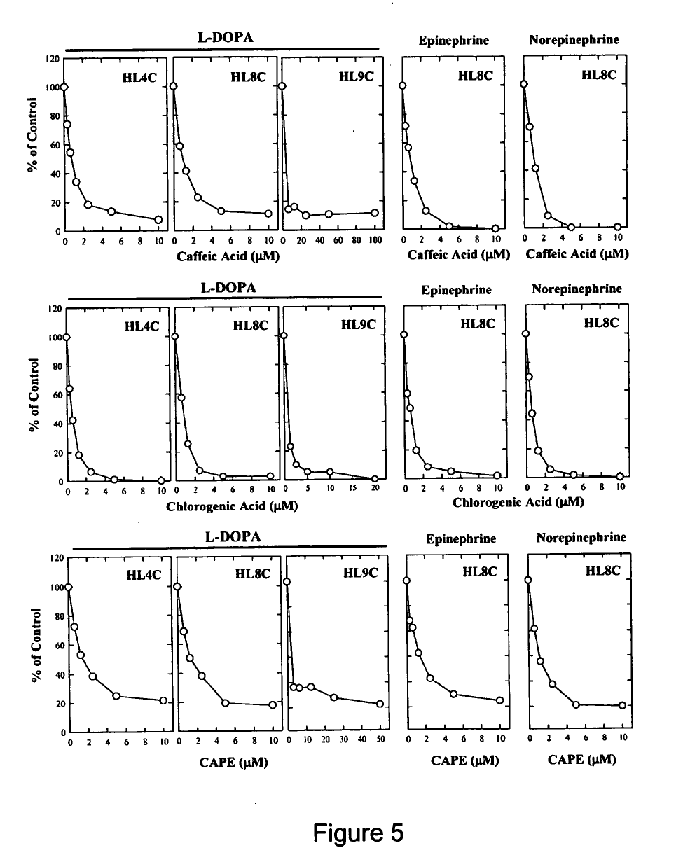

FIG. 5 demonstrates the metabolism data of coffee polyphenols (such as caffeic acid, caffeic acid pheethyl ester, and chlorogenic acid). As can be seen in FIG. 5, the coffee polyphenols are capable of strong inhibition of human COMT-mediated o-methylation of L-DOPA and other catecholamines. Their IC₅₀ values are 0.5-1.0 μM. FIG. 5 demonstrates the inhibition of human liver COMT-mediated o-methylation of catecholamines by caffeic acid (upper panels), chlorgenic acid (middle panels), and caffeic acid phenethyl ester (lower panels). The incubation mixture consisted of 10 μM catecholamine substrate, 250 μM [³H-methyl]AdoMet (containing 0.2 μCi), 0.25 mg/mL of human liver cytosolic protein, 1 mM dithiothreitol, 1.2 mM MgCl₂, and a dietary inhibitor (concentration as indicated) in a final volume of 0.25 mL tris-HCL buffer (10 mM, pH 7.4). Incubations were carried out at 37° C. for 10 min. Each point is the mean of duplicate determinations. Note that a total of three different human liver cytosolic preparations (HL4C, HL9C, HL8C) were tested.

These examples show that the metabolism rates of catecholamines in the liver are decreased in the presence of polyphenols, thereby allowing more of the catecholamine to reach the brain and effectively treat both the symptoms of Parkinson's disease, as well as the disease itself.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties.

The discussion of the references herein is intended merely to summarize the assertions made by their authors and no admission is made that any reference constitutes prior art. Applicants reserve the right to challenge the accuracy and pertinency of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part.

What is claimed is:

1. A method of treating Parkinson's disease comprising administering to a subject in need of treatment for Parkinson's disease at least one catecholamine, a decarboxylase inhibitor, and a polyphenol.

2. The method according to claim 1, wherein the at least one catecholamine is one or more of epinephrine, dopamine, and norepinephrine.

3. The method according to claim 1, wherein the at least one catecholamine is L-DOPA.

4. The method according to claim 1, wherein the decarboxylase inhibitor is one or both of cabidopa and benserazide.

5. The method according to claim 1, wherein the polyphenol is a dietary polyphenol.

6. The method according to claim 5, wherein the dietary polyphenol is a catechin.

7. The method according to claim 6, wherein the catechin is one or more of eatechin, epicatechin, epigallocatechin, and catechin gallates.

8. The method according to claim 7, wherein the polyphenol is a tea extract.

9. The method according to claim 8, wherein the tea extract is one or both of a green tea extract and a black tea extract.

10. The method according to claim 1, wherein the polyphenol is a coffee extract.

11. The method according to claim 1, wherein the catecholamine, the decarboxylase inhibitor, and the polyphenol are administered concurrently.

12. The method according to claim 1, wherein the catecholoamine, the decarboxlyase inhibitor, and the polyphenol are administered sequentially.

13. The method according to claim 1, wherein two of the catecholamine, the decarboxylase inhibitor, and the polyphenol are administered concurrently and the remaining component is administered separately.

14. The method according to claim 13, wherein the remaining component is administered prior to the administration of the other two components.

15. The method according to claim 13, wherein the remaining component is administered subsequent to the administration of the other two components.

16. The method according to claim 1, wherein the step of administering is an oral administration.

17. A pharmaceutical composition for treating Parkinson's disease, the composition comprising at least one catecholamine, at least one decarboxlyase inhibitor, and at least one polyphenol.

18. The pharmaceutical composition according to claim 17, wherein at least one of the catecholamine, the at least one decarboxylase inhibitor, and the at least one polyphenol is a discreet component.

19. The pharmaceutical composition according to claim 17, wherein at least two of the catecholamine, the at least one decarboxylase inhibitor, and at least one polyphenol are admixed.

20. The pharmaceutical composition according to claim 17, wherein the at least one catecholamine is selected from one or more of epinephrine, dopamine, and norepinephrine.

21. The pharmaceutical composition according to claim 17, wherein the at least one catecholamine is L-DOPA.

22. The pharmaceutical composition according to claim 17, wherein the at least one decarboxylase inhibitor is one or both of cabidopa and benserazide.

23. The pharmaceutical composition according to claim 17, wherein the at least one polyphenol is at least one dietary polyphenol.

24. The pharmaceutical composition according to claim 23, wherein the at least one dietary polyphenol is at least one catechin.

25. The pharmaceutical composition according to claim 24, wherein the at least one catechin is selected from the group consisting of catechin, epicatechin, epigallocatechin, and catechin gallates.

26. The pharmaceutical composition according to claim 17, wherein the at least one polyphenol is a tea extract.

27. The pharmaceutical composition according to claim 20, wherein the tea extract is one or both of a green tea extract and a black tea extract.

28. The pharmaceutical composition according to claim 17, wherein the polyphenol is a coffee extract.

29. The pharmaceutical composition according to claim 17, wherein the at least one polyphenol is present in a dosage amount of from
t 1 to about 100 mg/day kg.
